(19) United States
(12) Patent Application Publication
(10) Pub. No.: US 2010/0317703 A1
(43) Pub. Date: Dec. 16, 2010

(54) METHOD FOR INHIBITING BRASSININ OXIDASE

(75) Inventor: M. Soledade C. Pedras, Saskatoon (CA)

Correspondence Address: BERESKIN AND PARR LLP/S.E.N.C.R.L., s.r.l., 40 KING STREET WEST, BOX 401, TORONTO, ON, M5H 3Y2, CA

(73) Assignee: UNIVERSITY OF SASKATCHEWAN, Saskatoon (CA)

(21) Appl. No.: 12/815,578

(22) Filed: Jun. 15, 2010

Related U.S. Application Data
(60) Provisional application No. 61/186,956, filed on Jun. 15, 2009.

(51) Int. Cl. A01N 43/78; C07D 417/04; C12Q 1/26; C12N 1/00; A01P 3/00; A01N 43/38; A01N 43/80

(52) U.S. Cl. 514/365; 548/202; 435/25; 435/243; 514/415; 514/372

(57) ABSTRACT

The present disclosure relates to a method for inhibiting brassinin oxidase comprising treating a pathogen that is producing BO with an effective amount of selected Paldoxin compounds.

This application claims the benefit of Provisional Application No. 61/186,956, filed Jun. 15, 2009, the contents of which are herein incorporated by reference.

### FIELD OF THE DISCLOSURE

The present disclosure relates to a method for inhibiting brassinin oxidase. In particular, the method relates to inhibiting brassinin oxidase using specific phytoalexin detoxification inhibitors (paldoxins).

### BACKGROUND OF THE DISCLOSURE

Phytoalexins are antimicrobial secondary metabolites produced by plants in response to stress, including bacterial and fungal infection, heat, heavy metal salts, and UV radiation.¹ In general, cruciferous phytoalexins, produced by plants of the family Brassicaceae (common name crucifer), are biosynthesized from tryptophan and are produced as blends whose composition depends on the plant species and on the particular elicitor (stress factor).² Brassinin (1) is an essential phytoalexin due to its role as biosynthetic precursor of other cruciferous phytoalexins and its antimicrobial activity. The dithiocarbamate group of brassinin (1) is the toxophore responsible for its fairly broad antifungal activity.³ To the detriment of many agriculturally important crops, several pathogenic fungi of crucifers are able to overcome phytoalexins, such as brassinin¹ by detoxification.⁴ These detoxification reactions are induced only in the presence of the phytoalexin, but can rather quickly deprive the plant of its defense chemicals and facilitate an outcome favoring the fungal pathogen.

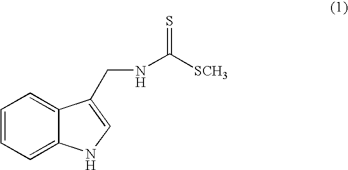

Cruciferous species include a wide variety of crops cultivated worldwide, for example, the oil seeds canola (Brassica napus and B. rapa L.) and rapeseed (B. napus and B. rapa) and many vegetables, such as rutabaga (B. napus ssp. napobrassica L.), turnip (B. rapa ssp. rapa L.) and cauliflower (B. oleraceae var. botrytis). Economically significant diseases of the oil seeds canola and rapeseed caused by fungi such as the “blackleg” fungi [Leptosphaeria maculans (asexual stage Phoma lingam) and L. biglobosa] are a global issue. L. maculans is a pathogen with well-established stratagems to invade crucifers, including production of specific enzymes in response to the phytoalexin, such as the phytoalexin brassinin (1), that detoxify essential plant defenses.⁴

The compounds 5-fluorocamalexin, 6-fluorocamalexin, 1-naphthalenylisothiazole, 1-naphthalenylisothiazole and camalexin have been studied as antifungal agents.^{5, 6}

### SUMMARY OF THE DISCLOSURE

The present disclosure relates to a method for inhibiting brassinin oxidase in plant pathogens.

Accordingly, the present disclosure includes a method for inhibiting brassinin oxidase (BO), comprising treating a pathogen that is producing BO, with an effective amount of:

(a) a compound of Formula I:

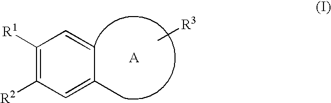

wherein,
A is a 5- or 6-membered unsaturated ring containing carbon and, optionally, nitrogen atoms;
R¹ and R² are independently or simultaneously selected from H, F, C₁-C₄alkyl, C₁, NO₂, OH, OC₁-C₄alkyl, NH₂, N(H)(C₁-C₄alkyl) and N(C₁-C₄alkyl)(C₁-C₄alkyl); and
R³ is a 5- or 6-membered unsaturated ring containing carbon and, optionally, nitrogen and/or sulfur atoms, that is bonded to a carbon atom in A, provided that when R³ is phenyl, R¹ and R² are not both H;
(b) a salt or solvate of the compound of Formula I;
(c) a compound selected from cyclobrassinin and isobrassinin;
(d) a salt or solvate of cyclobrassinin and isobrassinin; or
(e) a mixture of one or more of the compounds in (a), (b), (c) and (d).

In another embodiment of the disclosure, there is provided a use for inhibiting brassinin oxidase (BO) in a pathogen that is producing BO, of

(a) a compound of Formula I:

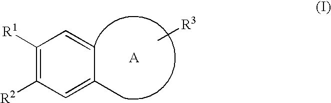

wherein,
A is a 5- or 6-membered unsaturated ring containing carbon and, optionally, nitrogen atoms;
R¹ and R² are independently or simultaneously selected from H, F, C₁-C₄alkyl, C₁, NO₂, OH, OC₁-C₄alkyl, NH₂, N(H)(C₁-C₄alkyl) and N(C₁-C₄alkyl)(C₁-C₄alkyl); and
R³ is a 5- or 6-membered unsaturated ring containing carbon and, optionally, nitrogen and/or sulfur atoms, that is bonded to a carbon atom in A, provided that when R³ is phenyl, R¹ and R² are not both H;
(b) a salt or solvate of the compound of Formula I;
(c) a compound selected from cyclobrassinin and isobrassinin;
(d) a salt or solvate of cyclobrassinin and isobrassinin; or
(e) a mixture of one or more of the compounds in (a), (b), (c) and (d).

Also included in the present disclosure are agricultural kits comprising

(I) one or more compounds selected from:
(a) a compound of Formula I:

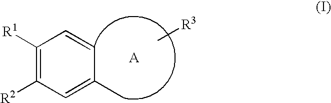

wherein,
A is a 5- or 6-membered unsaturated ring containing carbon and, optionally, nitrogen atoms;
R¹ and R² are independently or simultaneously selected from H, F, C₁-C₄alkyl, C₁, NO₂, OH, OC₁-C₄alkyl, NH₂, N(H)(C₁-C₄alkyl) and N(C₁-C₄alkyl)(C₁-C₄alkyl); and
R³ is a 5- or 6-membered unsaturated ring containing carbon and, optionally, nitrogen and/or sulfur atoms, that is bonded to a carbon atom in A, provided that when R³ is phenyl, R¹ and R² are not both H;
(b) a salt or solvate of the compound of Formula I;
(c) a compound selected from cyclobrassinin and isobrassinin; and
(d) a salt or solvate of cyclobrassinin and isobrassinin,
said compounds, salts and/or solvates being optionally combined with one or more agriculturally acceptable carriers or agriculturally acceptable excipients, and
(II) instructions for use of the one or more compounds to inhibit brassinin oxidase (BO) in a pathogen that is producing BO.

The enzyme involved in the oxidative detoxification of brassinin (1) by L. maculans, brassinin oxidase (BO), was purified and shown to catalyze the transformation of brassinin (1) to indole-3-carboxaldehyde (3, Scheme 1), a nonantifungal metabolite. This transformation appears to have no counterpart in the microbial metabolism of dithiocarbamates, despite the wide use of dithiocarbamates as fungicides for many decades.⁷ BO was stable and appeared to exhibit substrate specificity.

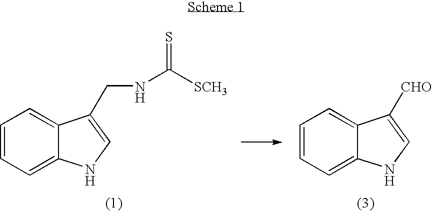

Accordingly, in another embodiment of the disclosure, there is provided a use of a purified and isolated BO enzyme for the identification of inhibitors of the BO.

In another embodiment of the disclosure, there is also provided a method for assaying and identifying inhibitors of the BO comprising:

i) contacting a purified and isolated BO with a substrate in the presence of one or more test substances; and

ii) determining an amount of conversion of the substrate in the presence of the one or more test substances compared to a control,

wherein if conversion of the substrate is less in the presence of the one or more test substances compared to the control, then the one or more test substances are inhibitors of BO.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### DETAILED DESCRIPTION OF THE DISCLOSURE

### Definitions

The term “C₁₋ₙalkyl” as used herein means a straight and/or branched chain, saturated alkyl group containing from one to “n” carbon atoms and includes (depending on the identity of n) methyl, ethyl, propyl, isopropyl, n-butyl, s-butyl, isobutyl and t-butyl, where the variable n is an integer representing the largest number of carbon atoms in the alkyl radical.

The term “OC₁₋ₙalkyl” as used herein means a straight and/or branched chain, saturated alkoxy group containing from one to “n” carbon atoms and includes (depending on the identity of n) methoxy, ethoxy, propoxy, isopropoxy, n-butoxy, s-butoxy, isobutoxy and t-butoxy, where the variable n is an integer representing the largest number of carbon atoms in the alkyl radical.

The term “unsaturated” as used herein means that the referenced group contains at least one unsaturated bond. An “unsaturated” bond is a double or triple bond. Suitably the unsaturated bond is a double bond. Unsaturated rings include non-aromatic and aromatic rings.

Cyclobrassinin is a compound of the formula:

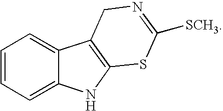

Isobrassinin is a compound of the formula:

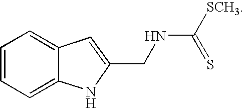

Thiabendazole is a compound of the formula:

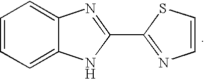

Camalexin is a compound of the Formula:

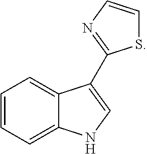

The term “salt” as used herein means any plant compatible organic or inorganic acid or basic addition salt of any neutral compound. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline, alkylammonias or ammonia. Either the mono or di-acid/base salts can be formed, and such salts may exist in either solvated or substantially anhydrous form. In general, salts are more soluble in water and various hydrophilic organic solvents, and generally demonstrate higher melting points in comparison to their free base forms. The selection of the appropriate salt will be known to one skilled in the art. The formation of a desired compound salt is achieved using standard techniques. For example, the neutral compound is treated with an acid or base in a suitable solvent and the formed salt is isolated by filtration, extraction or any other suitable method

The term “solvate” as used herein means a compound or its pharmaceutically acceptable salt, wherein molecules of a suitable solvent are incorporated in the crystal lattice. A suitable solvent is plant tolerable at the dosage administered. Examples of suitable solvents are ethanol, water and the like. When water is the solvent, the molecule is referred to as a “hydrate”. The formation of solvates of compounds will vary depending on the compound and the solvate. In general, solvates are formed by dissolving the compound in the appropriate solvent and isolating the solvate by cooling or using an antisolvent. The solvate is typically dried or azeotroped under ambient conditions.

As used herein, the phrase “effective amount” means an amount effective of one or more compounds, at dosages and for periods of time necessary to achieve the desired result. For example in the context of inhibiting BO activity, an effective amount is an amount that, for example, inhibits BO activity compared to the response obtained without administration or use of the compound(s). Effective amounts may vary according to factors such as the identity of the pathogen, the identity of the plant, the identity of the one or more compounds and the environmental conditions, and the like, but can nevertheless be routinely determined by one skilled in the art.

As used herein, to “inhibit” or “suppress” or “reduce” a function or activity, such BO activity, is to reduce the function or activity when compared to a control, for example, otherwise same conditions except for a condition or parameter of interest, or alternatively, as compared to another condition. The terms “inhibitor” and “inhibition”, in the context of the present disclosure, are intended to have a broad meaning and encompass compounds which directly or indirectly (e.g., via reactive intermediates, metabolites and the like) act on BO.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### METHODS OF THE DISCLOSURE

Selective BO inhibitors have been developed to prevent fungal detoxification of brassinin (1) in infected plants. Paldoxin is a term coined to address this new class of fungal enzyme inhibitors (phyloalexin detoxification inhibitors), that conceptualizes a new generation of chemicals designed, for example, for sustainable treatments of agricultural crops. Paldoxins are envisioned to inhibit unique metabolic reactions in fungal phytopathogens and, therefore, are less likely to affect non-targeted organisms and, thus, are expected to have minimal impact on cultivated ecosystems. Having, on hand, BO that has been purified to homogeneity has allowed the identification, for the first time, compounds that will selectively inhibit this enzyme and thereby inhibit the detoxification of brassinin oxidase by plant pathogens. This allows the plant to maintain its defense mechanism against pathogens that produce the detoxifying enzyme, BO, in response to the plant's production of brassinin.

The present disclosure accordingly relates to a method for inhibiting brassinin oxidase (BO), comprising treating a pathogen that is producing BO, with an effective amount of

(a) a compound of Formula I:

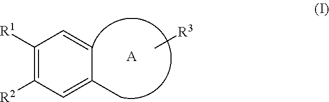

wherein,
A is a 5- or 6-membered unsaturated ring containing carbon and, optionally, nitrogen atoms;
R¹ and R² are independently or simultaneously selected from H, F, C₁-Cₐalkyl, C₁, NO₂, OH, OC₁-C₄alkyl, NH₂, N(H)(C₁-C₄alkyl) and N(C₁-C₄alkyl)(C₁-C₄alkyl); and
R³ is a 5- or 6-membered unsaturated ring containing carbon and, optionally, nitrogen and/or sulfur atoms, that is bonded to a carbon atom in A, provided that when R³ is phenyl, R¹ and R² are not both H;
(b) a salt or solvate of the compound of Formula I;
(c) a compound selected from cyclobrassinin and isobrassinin;
(d) a salt or solvate of cyclobrassinin and isobrassinin; or
(e) a mixture of one or more of the compounds in (a), (b), (c) and (d).

In another embodiment of the disclosure, there is provided a use for inhibiting brassinin oxidase (BO) in a pathogen that is producing BO, of

(a) a compound of Formula I:

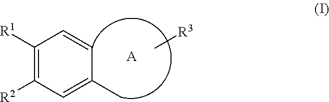

wherein,
A is a 5- or 6-membered unsaturated ring containing carbon and, optionally, nitrogen atoms;
R^{l} and R² are independently or simultaneously selected from H, F, Cl, NO₂, OH, OC₁-C₄alkyl, NH₂, N(H)(C₁-C₄alkyl) and N(C₁-C₄alkyl)(C₁-C₄alkyl); and
R³ is a 5- or 6-membered unsaturated ring containing carbon and, optionally, nitrogen and/or sulfur atoms, that is bonded to a carbon atom in A, provided that when R³ is phenyl, R¹ and R² are not both H;
(b) a salt or solvate of the compound of Formula I;
(c) a compound selected from cyclobrassinin and isobrassinin;
(d) a salt or solvate of cyclobrassinin and isobrassinin; or
(e) a mixture of one or more of the compounds in (a), (b), (c) and (d).

In an embodiment of the methods and uses of the disclosure, R¹ and R² in the compounds of Formula I are independently or simultaneously selected from H, F, C₁-C₂alkyl, C₁, NO₂, OH, OC₁-C₄alkyl, NH₂, N(H)(C₁-C₂alkyl) and N(C₁-C₂alkyl)(C₁-C₂alkyl). In a further embodiment, R¹ and R² are independently or simultaneously selected from H, F and OC₁-C₂alkyl. In another embodiment, R¹ and R² are independently or simultaneously selected from H, F and OC₁-C₂alkyl. In a further embodiment, R^{l} and R² are independently or simultaneously selected from H, F and OCH₃.

In an embodiment of the methods and uses of the disclosure, R³ is 5-membered unsaturated ring containing at least one nitrogen and/or sulfur atom. In a further embodiment, R³ is 5-membered unsaturated ring containing two double bonds one nitrogen and one sulfur atom. In yet another embodiment, R³ is a thiazole or isothiazole ring. In another embodiment, R³ is a 6-membered unsaturated ring optionally containing at least one nitrogen and/or sulfur atom. In a further embodiment, R³ is a 6-membered unsaturated ring optionally containing at least one nitrogen atom. In a further embodiment, R³ is a pyridine or a phenyl ring. In a further embodiment R³ is a phenyl ring.

In an embodiment of the methods and uses of the disclosure, A is 5-membered unsaturated ring containing at least one nitrogen atom. In further embodiment, A is a pyrrole ring. In another embodiment, A is a 6-membered unsaturated ring optionally containing at least one nitrogen atom. In a further embodiment, A is a pyridine or a phenyl ring. In a further embodiment A is a phenyl ring.

In an embodiment of the methods and uses of the present disclosure, the pathogen that is producing BO is treated with an effective amount of one or more compounds selected from:

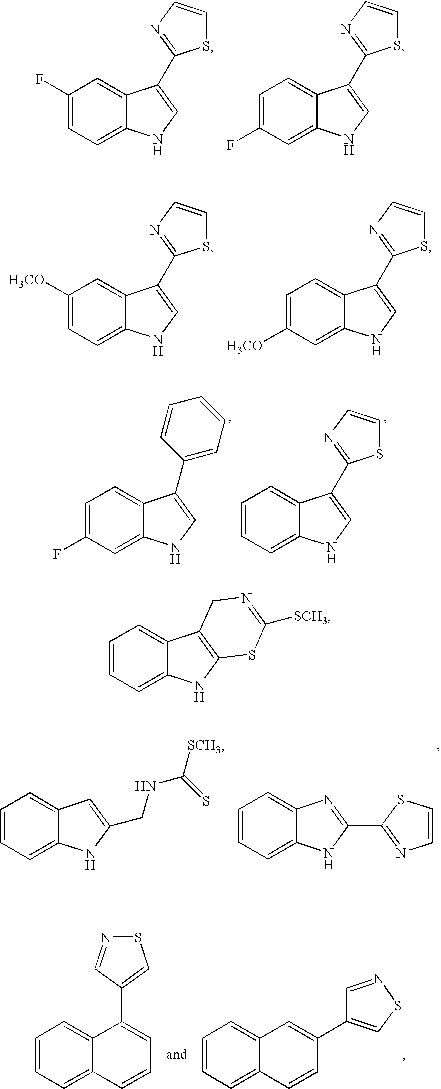

and salts and solvates thereof.

In the uses and methods of the present disclosure the compounds inhibit brassinin oxidase (BO) in a pathogen that is producing BO. In an embodiment, the inhibition of the pathogen is performed by administration of the compound(s) to a plant that has been infected with a pathogen and the pathogen has been induced to produce BO. BO is induced only when the pathogen has been challenged or exposed to brassinin. The pathogen may be any organism that produces BO in response to exposure to brassinin. In an embodiment, the pathogen is a fungus. In a further embodiment, the pathogen is Leptosphaeria maculans.

Also included in the present disclosure are agricultural kits comprising

(I) one or more compounds selected from:
(a) a compound of Formula I:

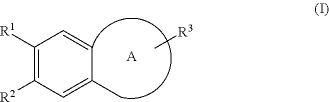

wherein,
A is a 5- or 6-membered unsaturated ring containing carbon and, optionally, nitrogen atoms;
R¹ and R² are independently or simultaneously selected from H, F, C₁-C₄alkyl, C₁, NO₂, OH, OC₁-C₄alkyl, NH₂, N(H)(C₁-C₄alkyl) and N(C₁-C₄alkyl)(C₁-C₄alkyl); and
R³ is a 5- or 6-membered unsaturated ring containing carbon and, optionally, nitrogen and/or sulfur atoms, that is bonded to a carbon atom in A, provided that when R³ is phenyl, R¹ and R² are not both H;
(b) a salt or solvate of the compound of Formula I;
(c) a compound selected from cyclobrassinin and isobrassinin; and
(d) a salt or solvate of cyclobrassinin and isobrassinin, said compounds, salts and/or solvates being optionally combined with one or more agriculturally acceptable carriers or agriculturally acceptable excipients, and
(II) instructions for use of the one or more compounds to inhibit brassinin oxidase (BO) in a pathogen that is producing BO.

In an embodiment of disclosure, the agricultural kits comprise a compound of Formula I in which R¹ and R² are independently or simultaneously selected from H, F, C₁-C₂alkyl, C₁, NO₂, OH, OC₁-C₄alkyl, NH₂, N(H)(C₁-C₂alkyl) and N(C₁-C₂alkyl)(C₁-C₂alkyl). In a further embodiment, R¹ and R² are independently or simultaneously selected from H, F and OC₁-C₂alkyl. In another embodiment, the agricultural kits comprise a compound of Formula I in which R¹ and R² are independently or simultaneously selected from H, F and OC₁-C₂alkyl. In a further embodiment, the agricultural kits comprise a compound of Formula I in which R¹ and R² are independently or simultaneously selected from H, F and OCH₃.

In an embodiment of the disclosure, the agricultural kits comprise a compound of Formula I in which R³ is 5-membered unsaturated ring containing at least one nitrogen and/or sulfur atom. In a further embodiment, R³ is 5-membered unsaturated ring containing two double bonds and one nitrogen and one sulfur atom. In yet another embodiment, R³ is a thiazole or isothiazole ring. In another embodiment, R³ is a 6-membered unsaturated ring optionally containing at least one nitrogen and/or sulfur atom. In a further embodiment, R³ is a 6-membered unsaturated ring optionally containing at least one nitrogen atom. In a further embodiment, R³ is a pyridine or a phenyl ring. In a further embodiment R³ is a phenyl ring.

In an embodiment of the disclosure, the agricultural kits comprise a compound of Formula I in which, A is 5-membered unsaturated ring containing at least one nitrogen atom. In further embodiment, A is a pyrrole ring. In another embodiment, A is a 6-membered unsaturated ring optionally containing at least one nitrogen atom. In a further embodiment, A is a pyridine or a phenyl ring. In a further embodiment A is a phenyl ring.

In another embodiment, the agricultural kits of the present disclosure, comprise one or more compounds selected from:

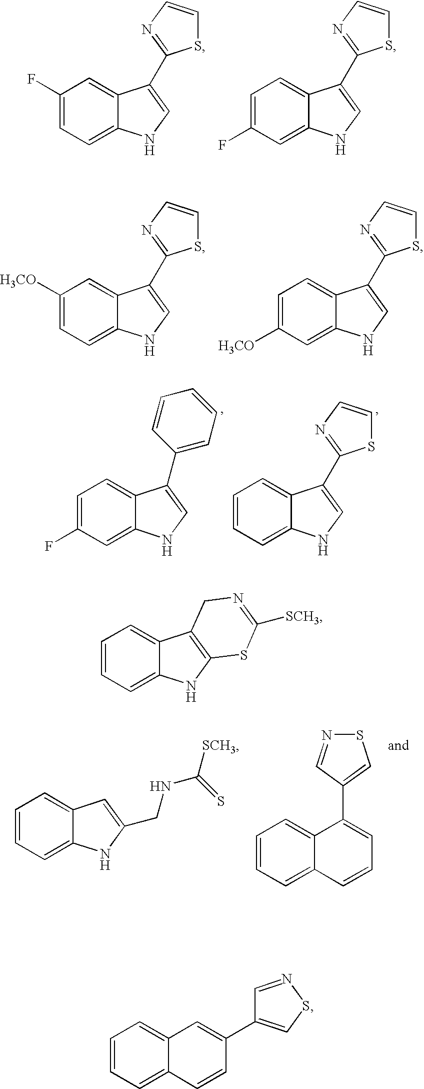

and salts and solvates thereof.

An agriculturally acceptable carrier may be solid, liquid or both. Solid carriers are, for example, selected from: mineral earth such as silicas, silica gels, silicates, talc, kaolin, montmorillonite, attapulgite, pumice, sepiolite, bentonite, limestone, lime, chalk, bole, loes, clay, dolomite, diatomaceous earth, calcite, calcium sulfate, magnesium sulfate, magnesium sulfate, magnesium oxide, sand, ground plastics, ferilizers such as ammonium sulfate, ammonium phosphate, ammonium nitrate, ureas, and crushed products of vegetable origin such as cereal meal, tree bark meal, wood meal and nutshell meal, cellulose powders, or other solid carriers. Optional excipients include organic solvents, water, surface active agents, and/or granular or particulate solid carriers.

Optionally included in the kits are other substances, such as a stabilizer, an emetic agent, a disintegrating agent, an antifoaming agent, a wetting agent, a dispersing agent, a binding agent, dye(s), fillers, carriers, surface active compounds (surfactants), and optionally solid and/or liquid auxiliaries and/or adjuvants such as wetters, adhesives, dispersants or emulsifiers. In an embodiment of the disclosure, such substances are included in a composition with the one or more compounds and carriers/excipients.

In another embodiment of the disclosure, there is provided a use of a purified and isolated BO enzyme for the identification of inhibitors of the BO.

In another embodiment of the disclosure, there is also provided a method for assaying and identifying inhibitors of the BO comprising:

i) contacting a purified and isolated BO with a substrate in the presence of one or more test substances; and

ii) determining an amount of conversion of the substrate in the presence of the one or more test substances compared to a control,

wherein if conversion of the substrate is less in the presence of the one or more test substances compared to the control, then the one or more test substances are inhibitors of BO.

In an embodiment, the control is an equivalent reactions set up, except for the absence of the one or more test substances. The one or more test substances can be any compound which one wishes to screen for inhibition of BO including, but not limited to, proteins (including antibodies), peptides, nucleic acids, fragments of proteins, peptides, carbohydrates, organic compounds, inorganic compounds and natural products. The one or more test compounds may also be, for example, a reaction mixture, library extracts, combinatorial libraries, bodily fluids and other samples that one wishes to test for the BO actitivity.

The following non-limiting examples are illustrative of the present disclosure:

### EXAMPLES

### Materials and Methods

All solvents were HPLC grade and used as such. Organic extracts were dried over anhydrous Na₂SO₄ and solvents removed under reduced pressure in a rotary evaporator. HPLC analysis was carried out with a high performance liquid chromatograph equipped with quaternary pump, automatic injector, and diode array detector (wavelength range 190-600 nm), degasser, and a Hypersil ODS column (5 μm particle size silica, 4.6 i.d.×200 mm), equipped with an in-line filter. Mobile phase: 75% H₂O-25% CH₃CN to 100% CH₃CN, for 35 min, linear gradient, and a flow rate 1.0 mL/min. Fourier transform IR spectra were obtained on a Bio-Rad FTS40 spectrometer in KBr. NMR spectra were recorded on 500 MHz spectrometers; δ values were referenced as follows: for ¹H (500 MHz), CDCl₃, 7.27 ppm; for ¹³C (125 MHz), CDCl₃, 77.23 ppm. Mass spectra (MS) were obtained on a VG 70 SE mass spectrometer using a solids probe or on a Q Star XL, Applied Biosystems.

All synthetic compounds were purified using flash column chromatography (FCC) on silica gel; satisfactory spectroscopic data identical to those previously reported were obtained for all previously reported compounds. Synthesis of brassinin and cyclobrassinin (1) was carried out as reported in the literature.² Thiabendazole was purchased from Sigma Aldrich, Oakville, Ontario, Canada.

### Example 1

### Compound I(a)

Compound I(a) was synthesized as reported in the literature.⁵

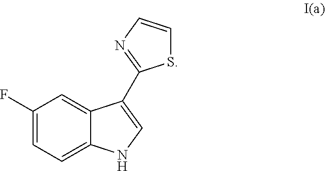

### Example 2

### Compound I(b)

Compound I(b) was synthesized as reported in the literature.⁹

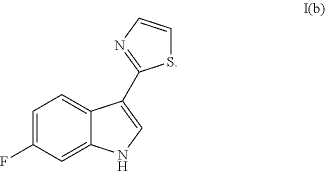

### Example 3

### Compound I(c)

Compound I(c) was synthesized as reported in the literature.¹⁰

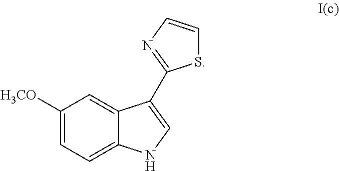

### Example 4

### Compound I(d)

Compound I(d) was synthesized as reported in the literature.¹⁰

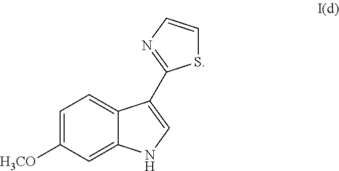

### Example 5

### Compound I(e)

Compound I(e) was synthesized as reported in the literature.¹¹

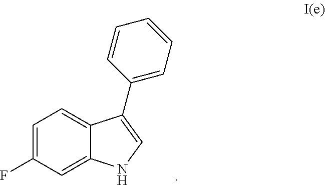

### Example 6

### Compound I(f)

Compound I(f) was synthesized as reported in the literature.⁶

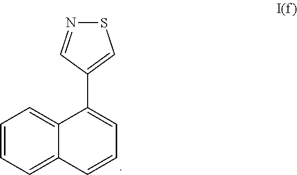

### Example 7

### Compound I(g)

Compound I(g) was synthesized as reported in the literature.⁶

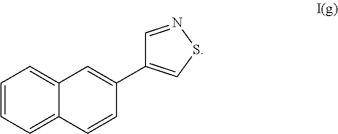

### Example 8

### Compound I(h)

Compound I(h) was synthesized as reported in the literature.¹⁰

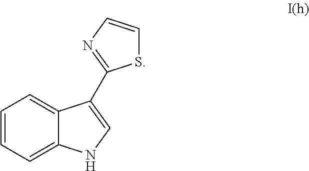

### Example 9

### Isobrassinin

Isobrassinin was synthesized as reported in the literature.¹³

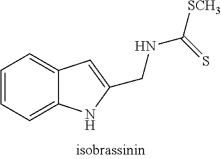

### Example 10

### Fungal Cultures

Liquid cultures of L. maculans (virulent isolate BJ-125, IBCN collection, AAFC) were handled as described in the literature.¹³ Fungal spores were subcultured on V8 agar under continuous light at 23±1° C.; after 15 days, fungal spores were collected aseptically and stored at −20° C.¹⁴ Liquid cultures were initiated by inoculating minimal media (100 mL)¹⁵ with fungal spores at 10⁷/mL in Erlenmeyer flasks, followed by incubation on a shaker under constant light at 23±1° C.

For purification of brassinin oxidase, 600 mL of 48 h old liquid cultures prepared as described above, were incubated with 3-phenylindole (19, 0.05 mM final concentration in cultures to induce BO) for an additional 24 h and then gravity filtered to separate mycelia from culture broth. The mycelia was stored at −20° C. up to 72 h and used to obtain protein extracts containing BO activity.

For analysis of BO induction, 72 h old liquid cultures (20 mL) were co-incubated with test compounds (final concentrations in culture 0.10, 0.20, and 0.50 mM), and after an additional incubation for 24 h, the mycelia were separated from the culture broth by filtration.

### Example 11

### Preparation of Protein Extracts for Analysis of Bo

Frozen mycelia (0.3-1.4 g) from L. maculans were suspended in ice-cold extraction buffer (1 mL) and ground (mortar) for 5 min. The extraction buffer consisted of diethanolamine(DEA, 25 mM, pH 8.3), 5% (v/v) glycerol, D,L-dithiothreitol (DTT, 1 mM), and 1/200 (v/v) protease inhibitor cocktail (P-8215, Sigma-Aldrich Canada). The homogenate was centrifuged at 4° C. for 30 min at 50000 g. The resulting supernatant was used for determination of specific activity of BO. Protein concentrations were determined as described by Bradford¹⁶ using the Coomassie Brilliant Blue method with BSA as a standard.

### Example 12

### BO Activity Assay

The reaction mixture contained DEA (20 mM, pH 8.3), DTT (1 mM), 0.1% (v/v) Triton X-100, brassinin (1, 1.0 mM), phenazine (0.50 mM), and protein extract (50-100 μL) in a total volume of 500 μL. The reaction was carried out at 24° C. for 20 min. A control reaction was stopped by the addition of EtOAc (2 mL) at t=0. The product was extracted with EtOAc (2 mL) and concentrated to dryness. The extract was dissolved in CH₃CN (200 μL) and analyzed by HPLC-DAD. The amounts of brassinin (1) and indole-3-carboxaldehyde (3) in the reaction assay were determined using calibration curves built with pure cyclobrassinin. One enzyme unit (U) is defined as the amount of the enzyme that catalyzes the conversion of one micromole of substrate per minute (μmol·min⁻¹=U).

### Example 13

### Chromatographic Purification of BO and Inhibitory Effect of Paldoxins

The purification of BO was performed in four steps as previously described.¹⁷ The purified enzyme was used for screening test compounds for inhibitory activity. To determine potential inhibitors of BO, inhibition experiments were carried out using brassinin (1, 0.10 mM final concentration) and test compounds (0.10 and 0.30 mM final concentrations). Standard deviation values for assays were determined from four independent experiments.

### Discussion

The inhibitory effects of test compounds on BO activity were tested at 0.10 mM and 0.30 mM using brassinin (1) as substrate (0.10 mM) and purified BO. Thiabendazole, a common fungicide, was used as the reference compound due to its commercial availability and BO inhibitory activity (ca. 25% at 0.30 mM).¹⁷ The concentrations of inhibitors were based on the Kₘ of BO for brassinin (1, 0.15 mM under the enzyme assay conditions), the natural substrate. Results of these enzymatic assays are summarized in Table 1.

Relative to camalexin, 5-methoxycamalexin I(c) was the most potent inhibitor of BO activity (ca. 72% at 0.30 mM), followed by 5-fluorocamalexin I(a) and 6-methoxycamalexin I(b), a natural phytoalexin² (ca. 63% at 0.30 mM). The inhibitory effect of the 6-fluoro derivative I(b) (46%, 0.30 mM) on BO activity was similar to that of camalexin (53%, 0.30 mM).

### Example 14

### Inducer Effect of Paldoxins

Mycelial cultures of L. maculans were incubated with the test compounds (0.10, 0.20, and 0.50 mM) for 24 h to evaluate potential induction of BO activity. The cultures were filtered, the mycelia were extracted with extraction buffer, and the resulting cell-free extracts were analyzed for BO activity using brassinin (1) as substrate. The total protein content of each cell-free extract was determined using a calibration curve built using BSA.

### Discussion

The results of these analyses are summarized in Table 2. In general, relative to control cultures all tested compounds induced BO activity, although there were substantial differences in the percentage of induction. For example, relative to controls, camalexin induced the highest amount of BO activity at 0.20 mM (6.9±0.3). Compound added to mycelial cultures

TABLE 1
Inhibitory Effect of Paldoxins on BO

| | | Inhibiton (%) | |
|---|---|---|---|
| Compound Name | Structure | 0.10 mM | 0.30 mM |
| Thiabendazole | 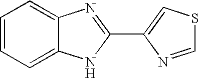 | 16 ± 3 | 25 ± 7 |
| Camalexin | 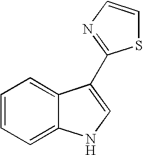 | 30 ± 4 | 53 ± 4 |
| Isobrassinin | 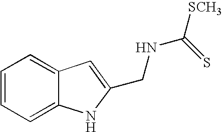 | 11 ± 5 | 23 ± 6 |
| Cyclobrassinin | 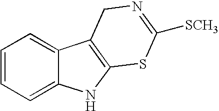 | 23 ± 6 | 37 ± 8 |
| 5-Fluorocamalexin I(a) | 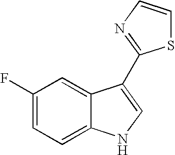 | 47 ± 5 | 63 ± 2 |
| 6-Fluorocamalexin I(b) | 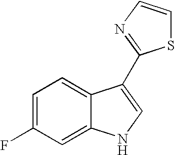 | 29 ± 10 | 46 ± 2 |
| 5-Methoxycamalexin I(c) | 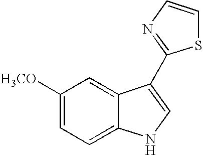 | 51 ± 4 | 72 ± 1 |
| 6-Methoxycamalexin I(d) | 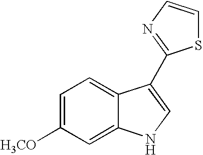 | 41 ± 6 | 63 ± 5 |
| 6-Fluoro-3- phenylindole I(e) | 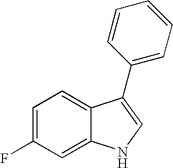 | 11 ± 4 | 17 ± 6 |
| 1-naphthalenyl- isothiazole I(f) | 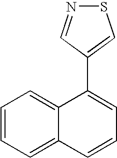 | 16 ± 4 | 21 ± 6 |
| 2-naphthalenyl- isothiazole I(g) | 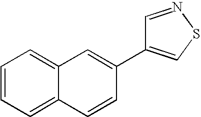 | 29 ± 2 | 42 ± 3 |
| Camalexin I(h) | 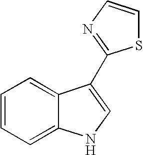 | 30 ± 4 | 53 ± 7 |

TABLE 2
Inducer and Antifungal Activity of Inhibitors

| Compound Added to | Concentration | Relative Specific | Relative |
|---|---|---|---|
| mycelial cultures (#) | (mM) | Activity of BO | Protein |
| control culture | | 1.0 | 1.0 |
| camalexin | 0.10 | 4.5 ± 0.5 | 0.28 |
| | 0.20 | 6.9 ± 0.3 | 0.24 |
| | 0.50 | 2.5 ± 0.1 | 0.10 |
| 5-fluorocamalexin I(a) | 0.10 | 5.0 ± 0.2 | 0.30 |
| | 0.20 | 4.9 ± 0.4 | 0.23 |
| | 0.50 | 7.9 ± 0.6 | 0.19 |
| 6-fluorocamalexin I(b) | 0.10 | 3.9 ± 0.1 | 0.36 |
| | 0.20 | 4.0 ± 0.2 | 0.28 |
| | 0.50 | 1.8 ± 0.3 | 0.24 |
| 5-methoxycamaiexin I(c) | 0.10 | 1.7 ± 0.3 | 0.28 |
| | 0.20 | 5.8 ± 0.6 | 0.24 |
| | 0.50 | 1.6 ± 0.3 | 0.13 |
| 6-methoxycamalexin I(d) | 0.10 | 2.3 ± 0.1 | 0.27 |
| | 0.20 | 3.7 ± 0.2 | 0.16 |
| | 0.50 | 4.0 ± 0.1 | 0.11 |
| thiabendazole | 0.10 | 1.9 ± 0.2 | 0.35 |
| | 0.20 | 4.1 ± 0.2 | 0.28 |
| | 0.50 | 4.9 ± 0.3 | 0.06 |
| 2-naphthalenyl- | 0.1 | 2.6 ± 0.2 | 0.26 |
| isothiazole I(f) | 0.2 | 8.5 ± 0.1 | 0.17 |
| | 0.5 | 16.3 ± 3.3 | 0.16 |

### FULL CITATIONS FOR DOCUMENTS REFERRED TO IN THE SPECIFICATION

(1) Bailey, J. A.; Mansfield, J. W. Phytoalexins; Bailey, Blackie and Son: Glasgow, U.K., 1982; P 334.
(2) Pedras, M. S. C.; Zheng, Q. A.; Sarma-Mamillapalle, V. K. The phytoalexins from Brassicaceae: Structure, biological activity, synthesis, and biosynthesis. Nat. Prod. Commun. 2007, 2, 319330.
(3) Pedras, M. S. C. The chemical ecology of crucifers and their fungal pathogens: Boosting plant defenses and inhibiting pathogen invasion. Chem. Rec. 2008, 8, 109-115.
(4) Pedras, M. S. C.; Ahiahonu, P. W. K. Metabolism and detoxification of phytoalexins and analogs by phytopathogenic fungi. Phytochemistry 2005, 66, 391-411.
(5) Pedras, M. S. C.; Liu, J. Designer phytoalexins: probing camalexin detoxification pathways in the phytopathogen Rhizoctonia solani. Org. Biomol. Chem. 2004, 2, 1070-1076
(6) Pedras, M. S. C.; Suchy, M. Design, synthesis and antifungal activity of inhibitors of brassinin detoxification in the plant pathogenic fungus Leptosphaeria maculans. Bioorg. Med. Chem. 2006, 14, 714-723.
(7) Pedras, M. S. C.; Gadagi, R. S.; Jha, M.; Sarma-Mamillapalle, V. K. Detoxification of the phytoalexin brassinin by isolates of Leptosphaeria maculans pathogenic on brown mustard involves an inducible hydrolase. Phytochemistry 2007, 68, 1572-1578.
(8) Russell, P. E. A century of fungicide evolution. J. Agric. Sci. 2005, 143, 11-25.
(9) Pedras, M. S. C.; Ahiahonu, P. W. K. Probing the phytopathogenic stem rot fungus with phytoalexins and analogues: unprecedented glucosylation of camalexin and 6-methoxycamalexin. Bioorg. Med. Chem. 2002, 10, 3307-3312.
(10) Ayer, W. A.; Craw, P. A; Ma, Y. T.; Miao, S. Synthesis of camalexin and related phytoalexins. Tetrahedron 1992, 48, 2919-2924.
(11) Pedras, M. S. C.; Hossain, M. Design, synthesis, and evaluation of potential inhibitors of brassinin glucosyltransferase, a phytoalexin detoxifying enzyme from Sclerotinia sclerotiorum. Bioorg. Med. Chem. 2007, 15, 5981-5996.
(12) Pedras, M. S. C.; Suchy, M.; Ahiahonu, P. W. K. Unprecedented chemical structure and biomimetic synthesis of erucalexin, a phytoalexin from the wild crucifer Erucastrum gallicum. Org. Biomol. Chem. 2006, 4, 691-701.
(13) Pedras, M. S. C.; Suchy, M. Detoxification pathways of the phytoalexins brassilexin and sinalexin in Leptosphaeria maculans: Isolation and synthesis of the elusive intermediate 3-formylindolyl2-sulfonic acid. argo Biomol. Chem. 2005, 3, 2002-2007.
(14) Pedras, M. S. C.; Khan, A. Q. Biotransformation of the brassica phytoalexin brassicanal A by the blackleg fungus. J. Agric. Food Chem. 1996, 44, 3403-3407.
(15) Pedras, M. S. C.; Biesenthal, C. J. Production of the host-selective toxin phomalide by isolation of Leptosphaeria maculans and its correlation with sirodesmin PL production. Can. J. Microbiol. 1998, 44,547-553.
(16) Bradford, M. M. A rapid and sensitive method for quantitation of microgram quantities of protein utilizing the principle of protein-dye-binding. Anal. Biochem. 1976, 72, 248-254.
(17) Pedras M. S. C.; Minic, Z. Jha, M. Brassinin oxidase, a fungal detoxifying enzyme to overcome a plant defense—Purification, characterization and inhibition. FEBS J. 2008, 275, 3691-3705.

We claim:

1. A method for inhibiting brassinin oxidase (BO), comprising treating a pathogen that is producing BO, with an effective amount of
(a) a compound of Formula I: 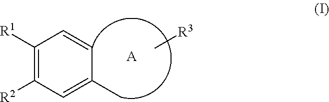
wherein,
A is a 5- or 6-membered unsaturated ring containing carbon and, optionally, nitrogen atoms;
R¹ and R² are independently or simultaneously selected from H, F, C₁-C₄alkyl, C₁, NO₂, OH, OC₁-C₄alkyl, NH₂, N(H)(C₁-C₄alkyl) and N(C₁-C₄alkyl)(C₁-C₄alkyl); and
R³ is a 5- or 6-membered unsaturated ring containing carbon and, optionally, nitrogen and/or sulfur atoms, that is bonded to a carbon atom in A, provided that when R³ is phenyl, R¹ and R² are not both H;
(b) a salt or solvate of the compound of Formula I;
(c) a compound selected from cyclobrassinin and isobrassinin;
(d) a salt or solvate of cyclobrassinin and isobrassinin; or
(e) a mixture of one or more of the compounds in (a), (b), (c) and (d).

2. The method of claim 1, R¹ and R² in the compounds of Formula I are independently or simultaneously selected from H, F, C₁-C₂alkyl, C₁, NO₂, OH, OC₁-C₄alkyl, NH₂, N(H)(C₁-C₂alkyl) and N(C₁-C₂alkyl)(C₁-C₂alkyl).

3. The method of claim 2, wherein R¹ and R² are independently or simultaneously selected from H, F and OC₁-C₂alkyl.

4. The method of claim 3, wherein R¹ and R² are independently or simultaneously selected from H, F and OC₁-C₂alkyl.

5. The method of claim 4, wherein R¹ and R² are independently or simultaneously selected from H, F and OCH₃.

6. The method of claim 1, wherein R³ in the compounds of Formula I is 5-membered unsaturated ring containing at least one nitrogen and/or sulfur atom.

7. The method of claim 1, wherein R³ is 6-membered unsaturated ring optionally containing at least one nitrogen atom.

8. The method of claim 1, wherein A in the compounds of Formula I is 5-membered unsaturated ring containing at least one nitrogen atom.

9. The method of claim 1, wherein A is a 6-membered unsaturated ring optionally containing at least one nitrogen atom.

10. The method of claim 1, wherein the pathogen that is producing BO is treated with an effective amount of one or more compounds selected from: 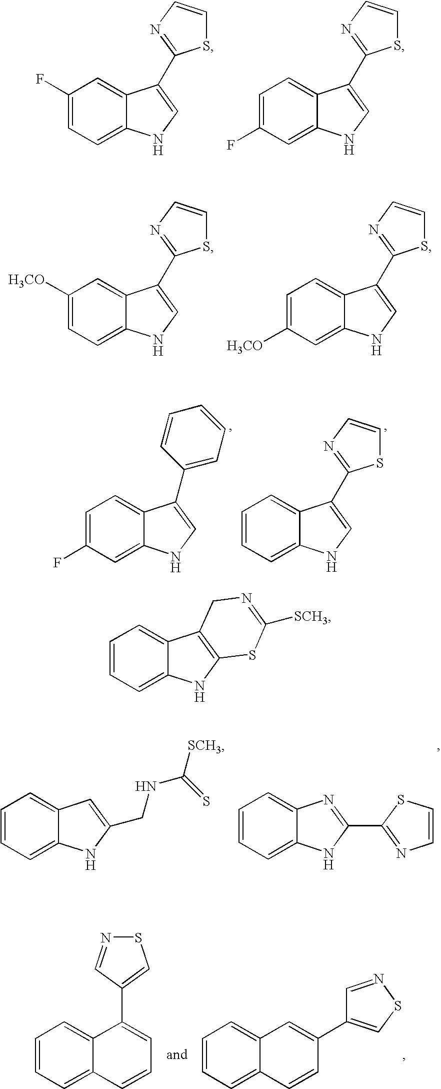
and salts and solvates thereof.

11. The method of claim 1, wherein the pathogen is any organism that produces BO in response to exposure to brassinin.

12. The method of claim 11, wherein the pathogen is a fungus.

13. The method of claim 12, wherein the pathogen is Leptosphaeria maculans.

14. An agricultural kit comprising
(I) one or more compounds selected from:
(a) a compound of Formula I: 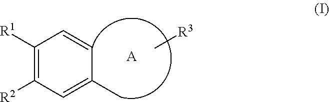
wherein,
A is a 5- or 6-membered unsaturated ring containing carbon and, optionally, nitrogen atoms;
R¹ and R² are independently or simultaneously selected from H, F, C₁-C₄alkyl, C₁, NO₂, OH, OC₁-C₄alkyl, NH₂, N(H)(C₁-C₄alkyl) and N(C₁-C₄alkyl)(C₁-C₄alkyl); and
R³ is a 5- or 6-membered unsaturated ring containing carbon and, optionally, nitrogen and/or sulfur atoms, that is bonded to a carbon atom in A, provided that when R³ is phenyl, R¹ and R² are not both H;
(b) a salt or solvate of the compound of Formula I;
(c) a compound selected from cyclobrassinin and isobrassinin; and
(d) a salt or solvate of cyclobrassinin and isobrassinin, said compounds, salts and/or solvates being optionally combined with one or more agriculturally acceptable carriers or agriculturally acceptable excipients, and
(II) instructions for use of the one or more compounds to inhibit brassinin oxidase (BO) in a pathogen that is producing BO.

15. The kit of claim 14, comprising a compound of Formula I in which R¹ and R² are independently or simultaneously selected from H, F, C₁-C₂alkyl, C₁, NO₂, OH, OC₁-C₄alkyl, NH₂, N(H)(C₁-C₂alkyl) and N(C₁-C₂alkyl)(C₁-C₂alkyl).

16. The kit of claim 15, wherein R¹ and R² are independently or simultaneously selected from H, F and OC₁-C₂alkyl.

17. The kit of claim 16, wherein R¹ and R² are independently or simultaneously selected from H, F and OC₁-C₂alkyl.

18. The kit of claim 17, wherein R¹ and R² are independently or simultaneously selected from H, F and OCH₃.

19. The kit of claim 14 comprising one or more compounds selected from: 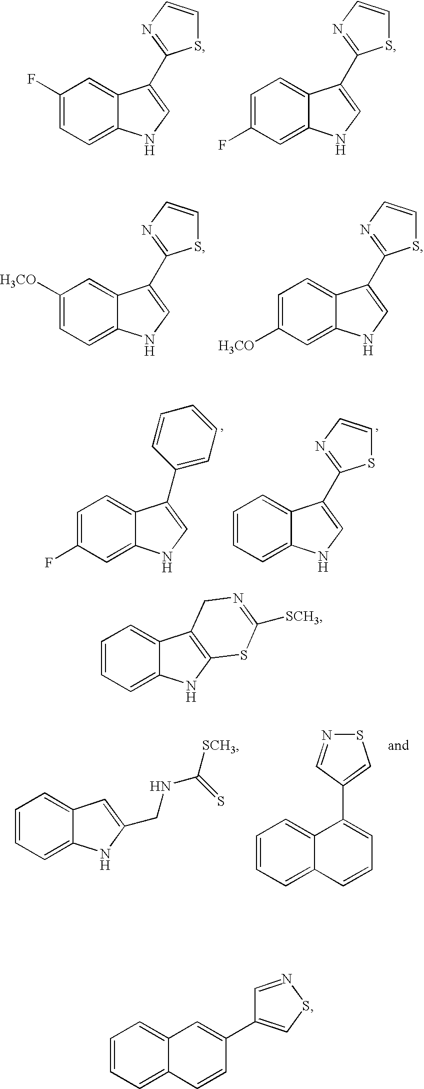
and salts and solvates thereof.

20. A method for assaying and identifying inhibitors of the BO comprising:
i) contacting a purified and isolated BO with a substrate in the presence of one or more test substances; and
ii) determining an amount of conversion of the substrate in the presence of the one or more test substances compared to a control,
wherein if conversion of the substrate is less in the presence of the one or more test substances compared to the control, then the one or more test substances are inhibitors of BO.
